(19) United States
(12) Patent Application Publication
(10) Pub. No.: US 2012/0264108 A1
(43) Pub. Date: Oct. 18, 2012

(54) Intracellular Molecular Delivery Based On Nanostructure Injectors

(75) Inventors: Xing Chen, Newton, MA (US); Carolyn R. Bertozzi, Berkeley, CA (US); Alexander K. Zettl, Kensington, CA (US)

(73) Assignee: THE REGENTS OF THE UNIVERSITY OF CALIFORNIA, Oakland, CA (US)

(21) Appl. No.: 12/521,098

(22) PCT Filed: Dec. 20, 2007
(86) PCT No.: PCT/US07/88323

§ 371 Date: Jul. 6, 2012

Related U.S. Application Data
(60) Provisional application No. 60/878,924, filed on Jan. 5, 2007.

(51) Int. Cl. C12M 1/34; C12N 5/07; C12N 15/89; C12Q 1/02; C12Q 1/70; B82Y 5/00

(52) U.S. Cl. 435/5; 435/288.7; 435/29; 435/375; 435/455; 977/904; 977/906; 977/745; 977/762

(57) ABSTRACT

There is disclosed a method and device for the delivery of molecules into individual cells. A device for injecting a biological molecule into a target cell comprises a microscopic tip attached to a mechanical scanning device for positioning the tip relative to the target cell and for moving the tip into the target cell; a nanostructure, such as a carbon nanotube, fixed on an end of the microscopic tip; and a biological molecule attached to the nanotube by a cleavable electrostatic or chemical linker linking the biomolecule to the nanotube, said chemical linker having a chemical linkage which is cleaved in an intracellular environment. The biological molecule may be one or more of proteins, nucleic acids, small molecule drugs, and optical labels, and combinations thereof. Exemplified are multiple walled carbon nanotubes to which a polycyclic aromatic compound is adsorbed, the aromatic compound having a side chain containing a cleavable disulfide linkage and a biotin functionality for coupling to a streptavidin-linked payload.

### CROSS-REFERENCE TO RELATED APPLICATIONS

This application claims priority to U.S. Provisional Patent Application 60/878,924, filed Jan. 5, 2007, hereby incorporated by reference in its entirety.

### STATEMENT OF GOVERNMENTAL SUPPORT

This invention was made with U.S. Government support under Contract Number DEACO2-05CH11231 between the U.S. Department of Energy and The Regents of the University of California for the management and operation of the Lawrence Berkeley National Laboratory. The U.S. Government has certain rights in this invention.

### REFERENCE TO SEQUENCE LISTING, COMPUTER PROGRAM, OR COMPACT DISK

None.

### BACKGROUND OF THE INVENTION

1. Field of the Invention

The present invention relates to the field of microinjection for introducing a substance, particularly peptides, nucleic acids, or other biologically active molecules into biological cells, as well as cell compartments such as plastids, cell nuclei, etc., as well as to an apparatus for performing this process.

2. Related Art

Cell microinjection—the direct-pressure injection of a solution into a cell through a glass capillary or micropipette—is an effective and reproducible method for introducing exogenous materials into cells. The technique, requiring little more than a sharp hollow needle to puncture the cell and an optical microscope to guide the process, has changed little since its inception in 1911 (1). Today, microinjection is broadly used as a valuable tool for the study of many different cell responses in a variety of systems. Nucleic acids, proteins, and even small molecules have been microinjected into relatively large objects, including frog eggs, cultured mammalian cells, mammalian embryos, and plant protoplasts and tissues (2). Microinjection, however, has several intrinsic limitations and drawbacks: i) There is a damaging effect produced by the introduction of the micropipette (with a micron scale tip) and by the pressure exerted by the injected fluid. ii) Cell types are usually limited to larger cells with tough membranes. Smaller cells such as bacteria are far less amenable to microinjection. iii) The limited spatial resolution of micromanipulation precludes targeting to a specific organelle other than nuclei within a cell.

The influx of nanotechnology has begun to impact the field of biotechnology (3). Recent efforts towards single cell study suggest the possibility of overcoming the limitations of microinjection by taking advantages of nanotechnology (4-8).

### PATENTS AND PUBLICATIONS

U.S. Pat. No. 6,063,629 to Knoblauch, issued May 16, 2000, entitled “Microinjection process for introducing an injection substance particularly foreign, genetic material, into procaryotic and eucaryotic cells, as well as cell compartments of the latter (plastids, cell nuclei), as well as nanopipette for the same,” discloses a nanopipette which has an external diameter of 0.05 to 0.2 μm, an internal holding diameter of 0.1 to 1.5 mm and a tip diameter of 0.025 to 0.3 μm and is filled with an injection substance and a heat-expandable substance. The capillary of the nano-pipette is then sealed with an adhesive, the pipette tip, with the aid of a microscope and a micromanipulator, is stuck into the desired plastids, bacterium or cell compartment/cell nucleus and the nanopipette is heated.

McKnight et al., “Intracellular integration of synthetic nanostructures with viable cells for controlled biochemical manipulation,” 2003 Nanotechnology 14 551-556, discloses the integration of vertically aligned carbon nanofibre (VACNF) elements with the intracellular domains of viable cells for controlled biochemical manipulation. Deterministically synthesized VACNFs were modified with either adsorbed or covalently-linked plasmid DNA and were subsequently inserted into cells.

Williams et al., “Controlled placement of an individual carbon nanotube onto a microelectromechanical structure,” App. Phys. Lett. 80 (14): 2574-2576 (2002) discloses a mechanical system whereby an individual multiwalled CNT (carbon nanotube) was retrieved from a cartridge by the AFM (atomic force microscope) tip, translated to a MEMS device, and placed thereon. While observing the AFM tip in the SEM, the authors controlled the motion of the tip to bring it down in contact with the CNTs on the cartridge, then slowly moved up and away from the cartridge. Upon removal of the tip from the cartridge, a single CNT had adhered to the tip, presumably through van der Waals forces. The CNT was approximately 3 μm long and 50-100 nm in diameter.

Wade et al., “Single-molecule Fluorescence and Force Microscopy Employing Carbon Nanotubes,” Nanotech 2003, 2003, 3, 317, discloses that AFM imaging with nanotube tips suitable for imaging dry samples with very high-resolution has been developed.

U.S. 20050191427 to Wade, et al., published Sep. 1, 2005, “Selective functionalization of carbon nanotube tips allowing fabrication of new classes of nanoscale sensing and manipulation tools,” discloses that pulsing in other gases than air, such as H₂ or N₂, will introduce different functionality to a carbon nanotube end on an AFM tip, allowing for an expansion of possible chemical coupling techniques. Covalent coupling chemistry of the carboxylate moiety with reactive amino species, with EDC (1-ethyl-3-(3-dimethylaminopropyl) carbodiimide) as a catalyst, allows for the covalent attachment of many types of organic and biological species via formation of amide bonds. Examples of molecules attached in this way include but are not limited to DNA, proteins and fluorophores.

Bottini et al. “Full-Length Single-Walled Carbon Nanotubes Decorated with Streptavidin-Conjugated Quantum Dots as Multivalent Intracellular Fluorescent Nanoprobes,” Biomacromolecules, 7 (8), 2259 -2263, 2006 (Web Release Date: Jun. 28, 2006) discloses the formation of a supramolecular luminescent nanoassembly composed of individual or small ropes of full-length, single-walled carbon nanotubes decorated with streptavidin-conjugated quantum dots.

### BRIEF SUMMARY OF THE INVENTION

The following brief summary is not intended to include all features and aspects of the present invention, nor does it imply that the invention must include all features and aspects discussed in this summary.

The present invention comprises, in one aspect, an apparatus for injecting a molecule into a target cell, where the target cell is selected specifically by the operator of the apparatus. The apparatus comprises a tip attached to a mechanical scanning device for positioning the tip relative to the target cell and for moving the tip into the target cell. The tip may be a microscope tip such as is known for use with scanning probe microscopes, but any mounting structure on a micron scale will suffice, provided that the structure is provided with controls for microscopic movement in three dimensions. The device will further comprise a nanostructure fixed on an end of the tip. The nanostructure may be one of several suitable structures, chosen to be rigid for cell penetration, adaptable to a linker, and generally inert to the intracellular environment. The linker is a chemical linker on the nanostructure for attaching a payload molecule to the nanostructure by the chemical linker, said chemical linker having a chemical linkage, which is cleaved in an intracellular environment to release the molecule inside the cell.

In particular, payload molecules may comprise proteins and nucleic acids and may further comprise optical labels attached thereto. The mechanical scanning device is a preferably a scanning probe microscope, such as an atomic force microscope, which has scanning and tapping capabilities. The preferred nanotube is a carbon nanotube, which may be an SWNT or MWNT, or even ropes or combinations thereof.

An important aspect of the present invention is the use of a chemical linker, which links the biological molecule to the nanotube so that it is released into the cell, preferably in multiple copies. In a preferred form, the linker is of the formula Ar—R—X—Y where Ar is an aryl compound, R is an alkyl linker, X is a cleavable functionality; and Y is an alkyl-linked binding group for binding to a biological molecule. Ar is a polycyclic aromatic hydrocarbon, such as anthracene, naphthalene or pyrene, for being adsorbed onto a carbon surface. R—X—Y represents an alkyl (as may be substituted or modified) containing a cleavable linkage X. A preferred cleavable linkage is the disulfide bond, —S—S—. An electrostatic linker may be used, which also is cleavable within the cell, by reversing polarity on a charged tip, which is attached to the nanostructure, and by taking advantage of the naturally occurring negative charge that exists in many cells.

The capability of the nanoinjector was demonstrated by injection of protein-coated quantum dots into live human cells. The protein was streptavidin, which links to a small molecule, biotin, on the linker. That is, the linker was attached (non-covalently) at one end to the nanostructure injector and at the other end (covalently) to biotin. The non-covalent bonding to the injector was of very high affinity, in this case, through π-π stacking. Single-particle tracking was employed to characterize the diffusion dynamics of injected quantum dots (which are fluorescent) in the cytosol. This new technique causes no discernible membrane or cell damage and can deliver a discrete number of molecules to the cell's interior without the requirement of a carrier solvent. The molecule to be delivered is not simply added to a solution and injected. It is delivered without addition of any other material. By selecting the conditions for attaching the molecule to be delivered to the nanostructure, a controlled, finite number of molecules can be delivered.

### BRIEF DESCRIPTION OF THE DRAWINGS

FIG. 1 is a schematic diagram showing an example of a nanoinjection system according to the present invention, with cell membrane (A) and an injector array (B);

FIG. 2 is a series of electron microscope images, namely a scanning electron microscope (SEM) image of a CNT-AFM tip; (B) transmission electron microscope (TEM) image of the tip region of the structure in (A); and (C) and (D) are TEM images of QDot® semi-conductor nanocrystal streptavidin conjugates attached to a CNT-AFM tip;

FIG. 3 is a schematic diagram showing functionalization of CNT-AFM tips; in 3A, semi-conductor nanocrystal-streptavidin conjugates were attached to CNT surfaces through a linker (molecule 1, FIG. 3C) containing a disulfide, showing steps i (adding disulfide linker) and ii (adding biomolecule); in 3B, step iii is as above, while step iv is adding linker molecule 2;

FIG. 4 is a series of reaction schemes showing the synthesis of linkers 1 and 2 (4A); 4B is a drawing of compound 3; 4C is a drawing of compound 1; and 4D is a drawing of linker 2; and

FIG. 5 is a graph showing diffusion dynamics of quantum dots inside the cytoplasm.

### DETAILED DESCRIPTION OF THE PREFERRED EMBODIMENT

### Definitions

Except to the extent defined otherwise, all technical and scientific terms used herein have the same meaning as commonly understood by one of ordinary skill in the art.

The term “biological molecule” means a protein, nucleic acid, small molecule label, a small molecule drug, or other compound that may be delivered to a cell interior.

The term “small molecule” means an organic or organometallic compound such as a traditional pharmaceutical compound (see e.g., Physician's Desk Reference, Medical Economics Company, Montvale, N.J. (1996) either approved for marketing or in development. Small molecules which are drugs have a specific biological effect, although not necessarily beneficial, and include research reagents such as neurotoxins, protein synthesis inhibitors, DNA crosslinkers, etc. used in biological research. The term “small molecule” also includes labels, such as fluorescent dyes, or radioactive compounds. A small molecule will generally have a molecular weigh below about 10 kD, often less than 1 kD. The small molecules used here as payloads are typically prepared by synthetic chemical techniques.

The term “target cell” means a biological compartment, which encloses an interior environment differing from its surroundings. The term specifically includes living cells such as prokaryotic, plant, insect, bacterial, yeast, and animal cells. The term also includes non-living cells of the above types; and cell-like structures such as viruses, micelles, liposomes, etc. In practicing the present methods, target cells may be used wherein specific organelles within the cell are injected. These organelles include the nucleus (and nucleolus), Gogi apparatus, rough ER, vesicles, microtubules, lysosomes, mitotic spindles, etc. For example, the present device, operating on a nanoscale, can deliver a payload to a eukaryotic 26S proteasome, which is about 15 nm long and 11.5 nm wide, with a hollow core. It is preferred that the target cell be living, since the present apparatus causes little if any damage to the cell membrane being pierced. The target cell may also be part of an organized tissue. The present apparatus and methods are well suited for small target cells such as bacteria (which are 0.1 to 10 μm in diameter).

The term “nanostructure” means a material having a length at least ten times its diameter, having a length up to about one mm, preferably between 10 micron and 10 nanometers; and a diameter between 0.5 nm and 100 nm, preferably 1-20 nm, and being mechanically rigid and chemically unreactive during operation of the present injection. The term “nanostructure” includes nanotubes, nanowires, and nanorods.

The term “nanotube” is used here in a broad sense to include: carbon nanotubes (CNTs) such as single-walled carbon nanotubes (SWNTs), multiwalled carbon nanotubes (MWNTs); and other forms of nanotubes which have uniform mechanical properties and are chemically inert to the attached linker and intracellular environment. For example, BC₂N or BN nanotubes, as described in Zettl, “Non-Carbon Nanotubes,” Adv. Mat. 8 (5):443-445 (1996). Gold, palladium and platinum nanotubes are also included. See, Yugang et al., “Metal nanostructures with hollow interiors,” Advanced Materials, 2003, vol. 15, no 7-8, pp. 641-646.

The term “SWNTs,” although predominantly having a single wall, are understood to include instances within a given sample of tubes having multiple walls in some cases. See, Flauhaut et al., “Synthesis of single-walled carbon nanotube-Co—MgO composite powders and extraction of the nanotubes,” J. Mater. Chem. 2000, vol. 10, no 2, pp. 249-252.

The term “MWNT” means a carbon multiwalled nanotube. MWNTs (like SWNTs) have a near perfect carbon tubule structure that resembles a sheet of sp² bonded carbon atoms rolled into a seamless tube. They are generally produced by one of three techniques, namely electric arc discharge, laser ablation and chemical vapor deposition. The arc discharge technique involves the generation of an electric arc between two graphite electrodes, one of which is usually filled with a catalyst metal powder (e.g., iron, nickel, cobalt), in a helium atmosphere. The laser ablation method uses a laser to evaporate a graphite target, which is usually filled with a catalyst metal powder too. The arc discharge and laser ablation techniques tend to produce an ensemble of carbonaceous material, which contain nanotubes (30-70%), amorphous carbon and carbon particles (usually closed-caged ones).

The term “nanowire” means an electrically conducting wire, which is extremely small Like conventional wires, nanowires can be made from a variety of conducting and semiconducting materials. Nanowires are less than 100 nanometers in diameter and can be as small as 3 nanometers. Typically nanowires are more than 1000 times longer than their diameter.

The term “nanorod” means a material having a rod-like morphology, with dimensions ranging from 1-100 nm. Nanorods may be synthesized from metals or semiconducting materials. Standard aspect ratios (length divided by width) are 3-5. Nanorods may be carbon (see, e.g., Science 10 Sep. 1999: Vol. 285. no. 5434, pp. 1719-1722); metal oxide (see U.S. Pat. No. 6,036,774); silicon carbide (see U.S. Pat. No. 5,997,832); metals and metal alloys such as copper, nickel and gold, see e.g., Salem et al., “Multi-component nanorods for vaccination applications,” Nanotechnology 16 484-487, 2005.

The term “chemical linker” means a molecular compound or complex that links a nanotube according to the present invention to the present biomolecule.

For example, it may have the chemical linker is of the formula Ar—R—X—Y where:

Ar is an aryl compound,

R is an alkyl linker,

X is a cleavable functionality; and

Y is an alkyl-linked binding group for binding to a biological molecule.

The cleavable functionality of X may be further defined as a cleavable linkage as a linkage cleaved within a target cell, at a rate causing cleavage of at least 10% of the bonds per hour, preferably cleavage of at least 10% of the bonds per minute, and as high as 100% per minute.

The term “alkyl” means a branched or unbranched, saturated or unsaturated acyclic hydrocarbon radical. Suitable alkyl radicals include, for example, methyl, ethyl, n-propyl, i-propyl, 2-propenyl (or allyl), vinyl, n-butyl, t-butyl, i-butyl (or 2-methylpropyl), etc. In particular embodiments, alkyls have between 1 and 20 carbon atoms, between 1 and 10 carbon atoms or between 1 and 5 carbon atoms. The term alkyl includes heteroalkyl, as defined below, “substituted alkyl,” which refers to an alkyl as just described in which one or more hydrogen atom bound to any carbon of the alkyl is replaced by another group such as a halogen, aryl, substituted aryl, cycloalkyl, substituted cycloalkyl, heterocycloalkyl, substituted heterocycloalkyl, halogen, alkylhalos (e.g., CF₃), hydroxy, phosphido, alkoxy, amino, thio, nitro, and combinations thereof. Suitable substituted alkyls include, for example, benzyl, trifluoromethyl and the like.

The term “heteroalkyl”means an alkyl as described above in which one or more carbon atoms to any carbon of the alkyl is replaced by a heteroatom selected from the group consisting of N, O, P, B, S, Si, Sb, Al, Sn, As, Se and Ge. The bond between the carbon atom and the heteroatom may be saturated or unsaturated. Thus, an alkyl substituted with a heterocycloalkyl, substituted heterocycloalkyl, heteroaryl, substituted heteroaryl, alkoxy, aryloxy, boryl, phosphino, amino, silyl, thio, or seleno is within the scope of the term heteroalkyl. Suitable heteroalkyls include cyano, benzoyl, 2-pyridyl, 2-furyl and the like.

The term “aryl” means an aromatic substituent, which may be a single aromatic ring or multiple aromatic rings, which are fused together, linked covalently, or linked to a common group such as a methylene or ethylene moiety. The aromatic ring(s) may include phenyl, naphthyl, anthracenyl, and biphenyl, among others. In particular embodiments, aryls have between 1 and 200 carbon atoms, between 1 and 50 carbon atoms or between 1 and 20 carbon atoms, including “substituted aryl,” which refers to aryl as just described in which one or more hydrogen atom bound to any carbon is replaced by one or more functional groups such as alkyl, substituted alkyl, cycloalkyl, substituted cycloalkyl, heterocycloalkyl, substituted heterocycloalkyl, halogen, alkylhalos (e.g., CF₃), hydroxy, amino, phosphido, alkoxy, amino, thio, nitro, and both saturated and unsaturated cyclic hydrocarbons which are fused to the aromatic ring(s), linked covalently or linked to a common group such as a methylene or ethylene moiety. The common linking group may also be a carbonyl as in benzophenone or oxygen as in diphenylether or nitrogen in diphenylamine.

The term aryl includes “heteroaryl,” which refers to aromatic or unsaturated rings in which one or more carbon atoms of the aromatic ring(s) are replaced by a heteroatom(s) such as nitrogen, oxygen, boron, selenium, phosphorus, silicon or sulfur. Heteroaryl refers to structures that may be a single aromatic ring, multiple aromatic ring(s), or one or more aromatic rings coupled to one or more non-aromatic ring(s). In structures having multiple rings, the rings can be fused together, linked covalently, or linked to a common group such as a methylene or ethylene moiety. The common linking group may also be a carbonyl as in phenyl pyridyl ketone. As used herein, rings such as thiophene, pyridine, isoxazole, pyrazole, pyrrole, furan, etc. or benzo-fused analogues of these rings are defined by the term “heteroaryl.”

“Substituted heteroaryl,” included in heteroaryl as just described including in which one or more hydrogen atoms bound to any atom of the heteroaryl moiety is replaced by another group such as a halogen, alkyl, substituted alkyl, aryl, substituted aryl, heteroaryl, substituted heteroaryl, alkoxy, aryloxy, boryl, phosphino, amino, silyl, thio, seleno and combinations thereof. Suitable substituted heteroaryl radicals include, for example, 4-N,N-dimethylaminopyridine.

The term “alkoxy” means the—OZ¹ radical, where Z¹ is selected from the group consisting of alkyl, substituted alkyl, cycloalkyl, substituted cycloalkyl, heterocylcoalkyl, substituted heterocycloalkyl, silyl groups and combinations thereof as described herein. Suitable alkoxy radicals include, for example, methoxy, ethoxy, benzyloxy, t-butoxy, etc. A related term is “aryloxy” where Z¹ is selected from the group consisting of aryl, substituted aryl, heteroaryl, substituted heteroaryl, and combinations thereof. Examples of suitable aryloxy radicals include phenoxy, substituted phenoxy, 2-pyridinoxy, 8-quinalinoxy and the like.

The term “amino” means the group—NZ¹Z², where each of Z¹ and Z² is independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof.

The term “thio” means the group—SZ¹, where Z¹ is selected from the group consisting of hydrogen, alkyl, substituted alkyl, cycloalkyl, substituted cycloalkyl, heterocycloalkyl, substituted heterocycloalkyl, aryl, substituted aryl, heteroaryl, substituted heteroaryl, alkoxy, aryloxy, silyl and combinations thereof.

The term “saturated” means a lack of double and triple bonds between atoms of a radical group such as ethyl, cyclohexyl, pyrrolidinyl, and the like.

The term “unsaturated” means the presence one or more double and/or triple bonds between atoms of a radical group such as vinyl, acetylide, oxazolinyl, cyclohexenyl, acetyl and the like.

### Overview

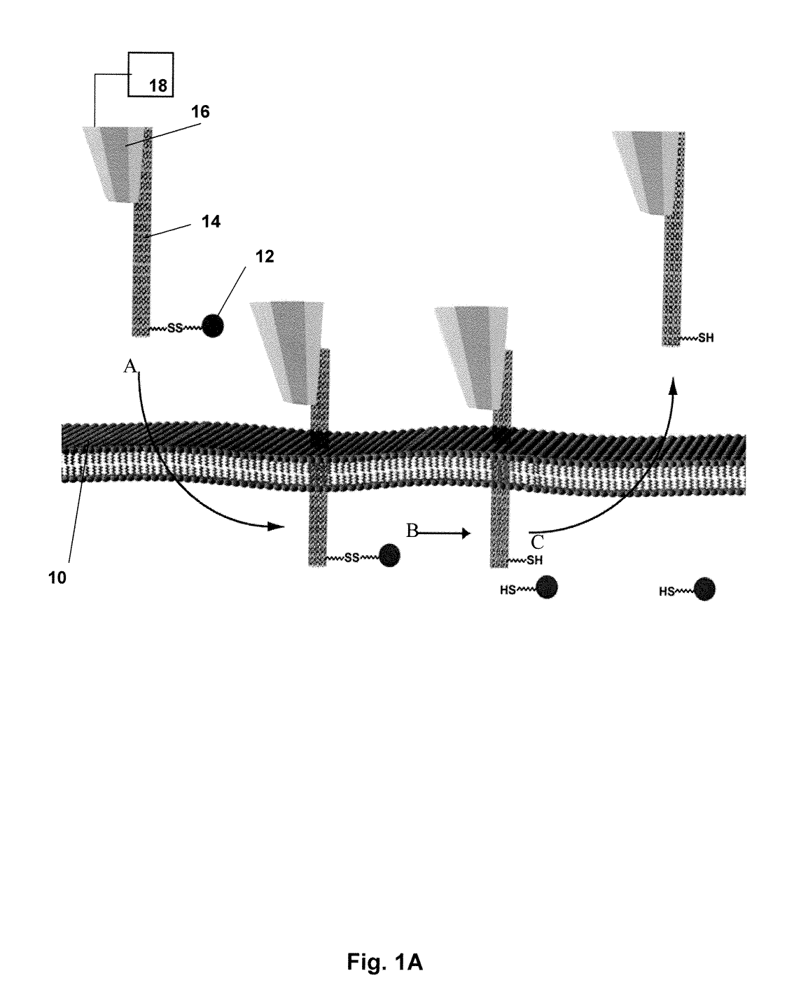

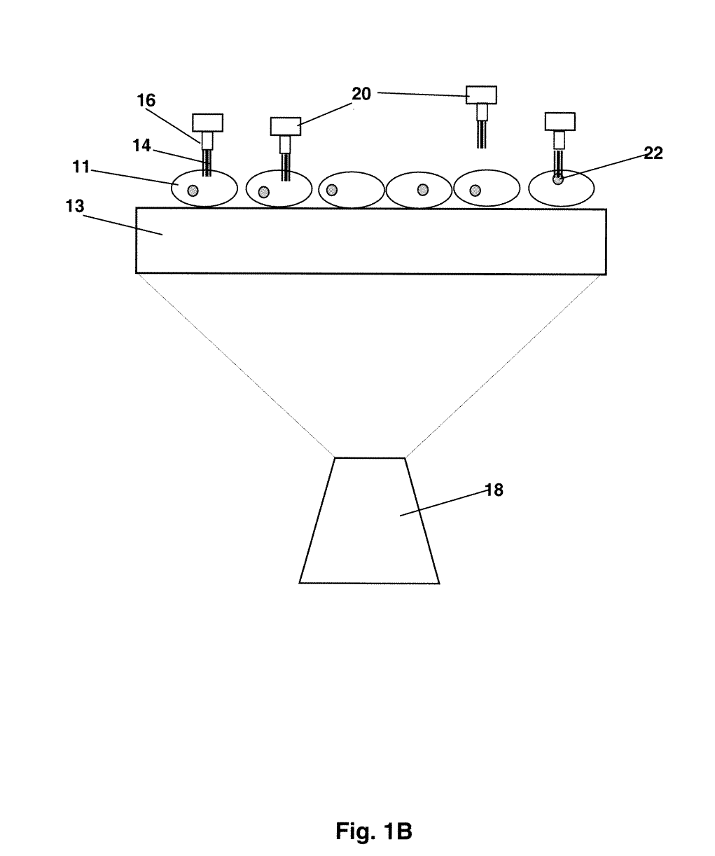

Described below is the construction of a versatile nanoscale cell injector so that the principle of microinjection can be expanded and applied more generally. Nanoinjection implies three essential components: a needle with nanoscale diameter, controllable loading and releasing of cargo, and a manipulator with nanoscale resolution for inserting and removing the needle. Herein, we describe the construction and successful operation of an exemplary cell injector with a CNT attached to an AFM tip serving as the nanometer scale needle and an AFM integrated with an inverted fluorescence microscope serving as the nanomanipulator (FIG. 1). Referring now to FIG. 1A, in there is represented a target cell having a lipid bilayer cell membrane 10, which can be part of a eukaryotic cell, having typical target organelles, e.g., nucleus, Gogi apparatus, rough ER, vesicles, mitotic spindles, etc., not shown. A biological molecule 12 is attached to a nanotube 14 through a disulfide linkage. The nanotube 14 is in turn attached to a microscope tip 16, in this case the tip of an AFM. The features of the cell are made visible and the tip may be guided through the aid of an inverted fluorescence microscope imaging the cell, as is shown at 18 (FIG. 1B). The imaging may be either on the same side or the opposite side of the cell. As shown by arrow A, the tip is lowered into the cell so that biological molecule 12 is inside the cell. Arrow B indicates that, after a time, the disulfide linkage is cleaved and the biological molecule (shown as thiolated) is released after the biological molecule is inserted into the target cell by movement of the AFM tip; in arrow C, the tip and nanotube are removed, leaving behind the biological molecule. A simple circuit 18 supplies a charge to the microscope tip and from there to the nanostructure attached to the tip. A circuit for applying a charge to the nanostructure to apply a bias voltage opposite of a charge on a payload is provided by a power supply within 18, and a fixed voltage connection, such as ground.

FIG. 1B shows an apparatus according to the present invention, where a plurality of cells 11 are arrayed on a substrate 13. The cells may be derived from a cell culture, a sample of tissue where the cells are to be further studies, a bacterial or plant culture, or other sources. The cell population may be homogeneous or heterogeneous. An optical microscope 18 is arranged to illuminate and image the cells, in this case from below, as is standard with an inverted microscope. An array of independently moveable injection assemblies is provided, comprising the nanostructure 14 and microscope tip 16 as in FIG. 1A. The microscope tip 16 is a moveable cantilever, as used in AFM microscopes. The assemblies 20 can be independently placed relative to cells selected through the use of microscope 18. An array of four injector assemblies is depicted, but any number can be fabricated. Each injector assembly can be directed through an XYZ manipulator, e.g., a micromanipulator capable of injection movements of less than 10 microns, preferably less than 1 micron, to a single cell, or to a cell organelle, such as nucleus 22. In addition, multiple assemblies can inject a single cell simultaneously.

CNTs, with needle-like geometry, and large Young's modulus and high tensile strength, are ideal nanoscale injectors for this purpose (9, 10). Their small diameters (1-20 nm) allow physical penetration of a cell's membrane without significant disruption of the cell's macrostructure. Indeed, such a piercing, which is on the scale of a single protein's diameter, should readily heal by lipid diffusion without perturbation of the cytoskeleton. Recent advances on covalent or non-covalent functionalization of CNTs have enabled the conjugations of CNTs with various biomolecules (11). The cargo can be chemically attached to the nanotube surface, rather than held in solution within the needle (FIG. 1A). A variety of linkages of cargo to CNTs such as disulfide and hydrazone bonds can be used to selectively release cargoes inside the cells (12, 13). In addition, this geometry avoids the requirement of a carrier or solvent and the addition of excess volume to the cell's cytosol during the injection process. In fact the payload molecule is delivered in pure form, with only a possible chemical modification to provide the cleavable linker. In some cases, e.g., cystine containing peptides, or DNA to be cleaved by nucleases, even this modification is not needed. The nanostructures used here are preferably attached to an SPM tip as a single tube, but the nanostructure injector may be comprised of axially aligned clusters of multiple tubes.

### CNT Injectors

Carbon nanotubes may consist of one tube of graphite, a one-atom thick single-wall nanotube (SWNT), or a number of concentric tubes called multiwalled nanotubes (MWNT). MWNTs (the embodiment exemplified below) for use in the present injector may be synthesized by the standard arc technique as described in Ebbesen et al., U.S. Pat. No. 5,641,466 issued Jun. 24, 1997, incorporated by reference specifically to describe a method for large-scale synthesis of carbon nanotube. These nanotubes have a near perfect carbon tubule structure that resembles a sheet of sp² bonded carbon atoms rolled into a seamless tube. They may be produced by several techniques. The arc discharge technique involves the generation of an electric arc between two graphite electrodes, one of which is usually filled with a catalyst metal powder (e.g., iron, nickel, cobalt), in a Helium atmosphere. The laser ablation method uses a laser to evaporate a graphite target, which is usually filled with a catalyst metal powder too. The arc discharge and laser ablation techniques tend to produce an ensemble of carbonaceous material, which contain nanotubes (30-70%), amorphous carbon and carbon particles (usually closed-caged ones). The nanotubes must then be extracted by some form of purification process before being manipulated into place for specific applications. The chemical vapor deposition process utilizes nanoparticles of metal catalyst to react with a hydrocarbon gas at temperatures of 500-900° C. A variant of this is plasma enhanced chemical vapor deposition in which vertically aligned carbon nanotubes can easily be grown. In these chemical vapor deposition processes, the catalyst decomposes the hydrocarbon gas to produce carbon and hydrogen. The carbon dissolves into the particle and precipitates out from its circumference as the carbon nanotube. Thus, the catalyst acts as a ‘template’ from which the carbon nanotube is formed, and by controlling the catalyst size and reaction time, one can easily tailor the nanotube diameter and length respectively to suit. Carbon tubes, in contrast to a solid carbon filament, will tend to form when the catalyst particle is ˜50 nm or less because if a filament of graphitic sheets were to form, it would contain an enormous percentage of ‘edge’ atoms in the structure. Alternatively, nanotubes may be prepared by catalytic pyrolysis of hydrocarbons as described by Endo, et al., in J. Phys. Chem. Solids, 54, 1841 (1993), or as described by Terrones, et al., in Nature, 388, 52 (1997) or by Kyotani, et al., in Chem. Mater., 8, 2190 (1996), the contents of all of which are incorporated by reference for describing nanotube preparation.

Suitable SWNTs are available from a number of sources. SWNTs are produced by laser vaporization (LV), electric-arc vaporization (AV) and by chemical vapor deposition (CVD). The LV and AV methods produce loose nanotubes, which are grown in the gas-phase from co-vaporized carbon and approximately 1% catalyst metal. CVD utilizes thermal decomposition of a mixture of carbon-containing and metal-catalyst-containing precursor gases (e.g., methane and ferrocene) above a hot substrate.

The bonding in the present carbon nanotubes can be considered as rolled-up graphene sheets (graphene is an individual graphite layer). There are three distinct ways in which a graphene sheet can be rolled into a tube, namely “armchair,” “zig-zag,” and “metallic,” all of which may be used here.

The strength of the covalent carbon-carbon bonds gives carbon nanotubes particular mechanical properties. The stiffness of the material, measured in terms of its Young's modulus, the rate of change of stress with applied strain, can be as high as 1000 GPa, which is approximately 5× higher than steel. The tensile strength, or breaking strain of nanotubes can be up to 63 GPa, around 50× higher than steel. These properties, coupled with the lightness of carbon nanotubes make them particularly preferred in the present injectors, where mechanical properties for penetrating biological materials is important.

It is also contemplated that the nanostructure used for injection may be a nanowire or nanorod. A suitable nanostructure useful in the present injector is a silicon nanowire, see, e.g., those described in Englander et al., “Local synthesis of silicon nanowires and carbon nanotubes on microbridges,” App. Phys. Lett. 32:4797-4799 (2003). The nanostructures as defined here may be doped or modified by the inclusion of other atoms.

Exemplary nanowires include aluminum, e.g., Ono et al., “Magnetic orderings in Al nanowires suspended between electrodes,” Applied Physics Letters—Jun. 23, 2003—Volume 82, Issue 25, pp. 4570-4572; those described in Geng et al., “Synthesis and optical properties of S-doped ZnO nanowires,” Synthesis and optical properties of S-doped ZnO nanowires,” Applied Physics Letters—Jun. 30, 2003—Volume 82, Issue 26, pp. 4791-4793; “Self-assembled growth of epitaxial erbium disilicide nanowires on silicon (001)” by Yong Chen, Douglas A. A. Ohlberg, Gilberto Medeiros-Ribeiro, Y. Austin Chang, and R. Stanley Williams in Applied Physics Letters, 76, p. 4004, June 2000, and silicon nanowires as described in Englander et al., “Local synthesis of silicon nanowires and carbon nanotubes on microbridges,” Applied Physics Letters—Jun. 30, 2003—Volume 82, Issue 26, pp. 4797-4799.

Useful nanorods may be carbon (see, e.g., Science 10 Sep. 1999: Vol. 285. no. 5434, pp. 1719 1722); metal oxide (see U.S. Pat. No. 6,036,774); silicon carbide (see U.S. Pat. No. 5,997,832); metals and metal alloys such as copper, nickel and gold, see e.g., Salem et al., “Multi-component nanorods for vaccination applications,” Nanotechnology 16 484-487, 2005. The nanostructure used for injection may be a combination of materials as well. See, e.g., Zhang et al. “Heterostructures of Single-Walled Carbon Nanotubes and Carbide Nanorods,” Science 285:1719-1722 (1999).

The nanostructures used for injection may be sized for particular applications and target cell types. Large cells with tough membranes can withstand large injectors, up to micron size. Generally, the injectors should be from 1 nm to 200 nm across and on the order of 500 nm to 1 mm or more in length.

The present device is implemented through a microscope tip attached to a mechanical scanning device for positioning the tip relative to the target cell. The mechanical scanning device refers to a movable tip or stage which allows the device to move on a microscopic scale relative to a target cell, which generally will be part of a sample of numerous cells or other, extracellular, material. The present microscope is preferably a scanning probe microscope. Scanning probe microscopy is based on the concept of scanning an extremely sharp tip (3-50 nm radius of curvature) across the object surface. The tip is mounted on a flexible cantilever, allowing the tip to follow the surface profile. When the tip moves in proximity to the investigated object, forces of interaction between the tip and the surface influence the movement of the cantilever. These movements are detected by selective sensors. Three exemplary scanning probe techniques are: Atomic Force Microscopy (AFM); Scanning Tunneling Microscopy (STM), which measures a weak electrical current flowing between tip and sample as they are held a very distance apart; and Near-Field Scanning Optical Microscopy (NSOM), which scans a very small light source very close to the sample. Detection of this light energy forms the image. NSOM can provide resolution below that of the conventional light microscope.

There are numerous variations on these techniques. AFM may operate in several modes, which differ according to the force between the tip and surface. In contact mode, the tip is usually maintained at a constant force by moving the cantilever up and down as it scans. In non-contact mode or intermittent contact mode (tapping mode) the tip is driven up and down by an oscillator.

Tapping Mode™ is a trademark of Digital Instruments. Tapping Mode imaging is implemented in ambient air by oscillating the cantilever assembly at or near the cantilever's resonant frequency using a piezoelectric crystal. The present technique, as carried out by the above-referenced XYZ manipulator, employs a variation of tapping, in which the tip is forced into the target cell and held there. In operation, one may adjust the tip to penetrate from about 10 nm (thickness of a cell membrane) to about 10-30 μm (diameter of a typical animal cell) into the target cell and be held in the target cell for at least a fraction of a second, preferably 10-60 seconds, up to several hours.

### Microscope Components

AFM tips are suitable for attachment of the present nanostructure. They are generally made of silicon or silicon nitride. They are relatively durable and present a hydrophobic surface to the sample. STM tips are made of mechanically formed or electrochemically-etched wire, usually noble metals or tungsten.

Numerous publications describe the design and implementation of atomic force microscopes useful in the present methods and device, for example, Atomic Force Microscopy, by Pier Carlo Braga, Davide Ricci, 1994, Humana Press.

For purposes of the present device, the microscopy aspect of scanning probe microscopy is not necessary. That is, necessary imaging can be done with light microscopy. However, it is advantageous in the present methods to be able to obtain both an SPM image and an optical image simultaneously. All that is needed is a positioning mechanism for placing a microscopic tip (i.e., commercial AFM tip or STM tip, typically having a pointed tip with a base radius of about 5-10-20 nm), bearing the present nanotube-payload conjugate, adjacent to a selected cell, and a mechanism for forcing the tip against the target cell surface. In the exemplified embodiment, the Asylum Research MFP-3D-BIO™ atomic force microscope was used. This device includes an inverted optical microscope, with capabilities for brightfield, Zernike phase contrast, or fluorescence microscopy, which can be used for acquiring a target cell and observing the results of the nanoinjected molecule. This device has a feature (shared with some other scanning probe microscopy devices) for force measurements that allows a user to push with the tip to conduct hardness and elasticity measurements, and to pull with the tip for protein folding, polymer stretching, receptor-ligand, and adhesion measurements. This pushing and pulling is used in the present injection and delivery of the described molecules.

### Delivery of PAYLOADS TO CELLS

The present device may be used to deliver a wide variety of molecules. These include nucleic acids, proteins, optical probes, magnetic probes, small molecule drugs, siRNA, smaller cells or organelles, and synthetic polymers.

Preferred protein payloads include antibodies, which may be specific to intracellular proteins or epitopes of interest. The present methods and materials may be applied to various kinds of antibodies, including, but not limited to, naturally occurring antibodies, single domain antibodies, hybrid antibodies, chimeric antibodies, single-chain antibodies, and antibody fragments that retain antigen binding specificity, intrabodies, and the like. Antibodies can be of any class (e.g., IgM, IgG, IgA, IgE; frequently IgG). Further exemplary antibodies include “univalent antibodies,” which are aggregates comprised of a heavy chain/light chain dimer bound to the Fe (i.e., constant) region of a second heavy chain. This type of antibody escapes antigenic modulation. See, e.g., Glennie et al. (1982) Nature 295:712 714. The term biological molecule also includes oligonucleotides and polynucleotides, preferably in the form of constructs which will effect or prevent expression of protein, such as siRNA, antisense DNA or RNA, expression vectors, etc. The labeling compounds will be either complexed with a small molecule drug, nucleic acid, peptide, etc., or may themselves be a payload, such as, for example, labeling specific intracellular targets. In this case, the labeling compound will ordinarily be one that is not taken up by a target cell, but must be injected.

For example, antibodies may be in the form of “Fab” fragments of antibodies. The “Fab” region refers to those portions of the heavy and light chains which are roughly equivalent, or analogous, to the sequences which comprise the branch portion of the heavy and light chains, and which have been shown to exhibit immunological binding to a specified antigen, but which lack the effector Fc portion. “Fab” includes aggregates of one heavy and one light chain (commonly known as Fab'), as well as tetramers containing the 2H and 2L chains (referred to as F(ab)₂), which are capable of selectively reacting with a designated antigen or antigen family. “Fab” antibodies may be divided into subsets analogous to those described above, i.e., “vertebrate Fab,” “hybrid Fab,” “chimeric Fab,” and “altered Fab.” Methods of producing “Fab” fragments of antibodies are known within the art and include, for example, proteolysis, and synthesis by recombinant techniques.

The present antibodies may take the form of intrabodies. Intracellular antibodies, or intrabodies, represent a class of neutralizing molecules with applications in gene therapy (vonMehren M, Weiner L M. (1996) Current Opinion in Oncology. 8: 493-498, Marasco, Wash. (1997) Gene Therapy. 4: 11-15, Rondon I J, Marasco Wash. (1997) Annual Review of Microbiology. 51: 257-283). Intrabodies can bind to specific targets in cells. They are normally single chain Fv fragments comprising variable domains of the immunoglobulin heavy (VH) and light chains (VL). It has been demonstrated that single VH domains have excellent intracellular properties of solubility, stability and expression within the cells of higher organisms and can exhibit specific antigen recognition in vivo. See Tanaka et al., “Single domain intracellular antibodies: A minimal fragment for direct in vivo selection of antigen-specific intrabodies,” J. Mol. Biol. 2003, vol. 331, no 5, pp. 1109-1120. Intrabodies may be delivered by the present nanoinjector as expressed proteins, or, as is known, in the form of genetic constructs, where they move to the nucleus and are there expressed. An example of an intrabody for diagnosis and treatment is given in “Diagnosis and treatment of malignant neoplasms,” U.S. Pat. No. 6,812,206, relating to an antibody which binds to a human aspartyl (asparaginyl) beta-hydroxylase (HAAH) polypeptide.

Preferred nucleic acids include RNA molecules such as the above-referenced siRNA, and other types of RNA used in RNA interference, as well as antisense RNA, and sense RNA. DNA may also be delivered.

### Applications

The present nanoinjector and methods for delivering molecules to individual cells, or individual compartments within cells, may be used to perform diagnostic tests on cells from patients. Only a single cell is necessary for testing. Many proteins involved in diseases are not secreted, but rather, are found only within cells. While one can assay these proteins indirectly through gene expression studies, it would be desirable to be able to assay the levels of these proteins directly. For example, see, Yan et al., “An intracellular protein that binds amyloid-β peptide and mediates neurotoxicity in Alzheimer's disease,” Nature 389 (6652):689-695 (1997). As disclosed there, amylold-β binds an intracellular polypeptide known as ERAB, thought to be a hydroxysteroid dehydrogenase enzyme, which is expressed in normal tissues, but is overexpressed in neurons affected in Alzheimer's disease. ERAB immunoprecipitates with amylold-β, and when cell cultures are exposed to amyloid-β, ERAB inside the cell is rapidly redistributed to the plasma membrane. Thus, for example, nanoinjection of amyloid—β could be used to measure ERAB as it is translocated to the cell membrane.

Cleavable linkers, discussed below, may be adapted to specific applications. For example, acid labile linkages may be used to deliver molecules to specific, acidic compartments under study, such as lysosomes. See, Cardelli et al., “Role of acidic intracellular compartments in the biosynthesis of Dictyostelium lysosomal enzymes. The weak bases ammonium chloride and chloroquine differentially affect proteolytic processing and sorting,” J. Biol. Chem., Vol. 264, Issue 6, 3454-3463, February, 1989.

Restriction enzymes in the nucleus or otherwise expressed in the target cell may be used to cleave oligonucleotide cleavable linkers.

Various peptide linkers may be designed for cleavage by specific intracellular proteases, see, e.g., Greene et al., “Enkephalin analog prodrugs: assessment of in vitro conversion, enzyme cleavage characterization and blood-brain barrier permeability,” Pharm. and Exp. Ther. Volume 277, Issue 3, pp. 1366-1375, Jun. 1, 1996.

### Cleavable Linkages

The chemical linker used to attach the payload to the nanoinjector will be tailored to bind to the nanostructure used for injection, as described above. In the present manufacturing method, one exposes a nanotube tip to a composition(s) having cleavable linkers and biological molecules. In this way, a number of biological molecules (between, e.g., 1 and 1 million, preferably on the order of thousands) are adsorbed onto a nanotube tip, and released only within a cell. The present cleavable linkages are cleavable intracellularly, rather than on the cell surface, as in the case of many immunoconjugates, but the intracellular environment contains a number of factors in common with those utilized in the design of immunoconjugate cleavage. For example, intracellular proteases or phosphodiesterases may be used to cleave linkages. Also, certain exogenous, membrane permeable elements may be added to cells to cause a cleavage reaction. Cheng et al. “See Cleavage of the Pt—S bond of thiolated terpyridine-platinum(ii) complexes by copper(ii) and zinc(ii) ions in phosphate buffer,” Chem. Comm. 1998:253-254 (1998). For an example of a proteolytic cleavage site, see Cook et al. “Biologically Active Interleukin 2-Ricin A Chain Fusion Proteins May Require Intracellular Proteolytic Cleavage To Exhibit a Cytotoxic Effect,” 1993, 4, 440-447.

As described in the above-cited U.S. Pat. No. 7,097,835, such cleavable linkers include, but are not limited to, the acid labile linkers described in U.S. Pat. No. 5,144,011. That patent describes cleavable linkers where each pair of cis-carboxyl groups formed by hydrolyzing a maleic anhydride is believed to comprise a suitable location for bonding a passenger molecule or a carrier molecule. Therefore, numerous passenger molecules may be bonded to a single polymeric spacer molecule. This allows for the transport of numerous passenger molecules to a desired acidic region, and for the release of large numbers of passenger molecules by the conjugate in that region, thereby increasing the efficiency of the conjugate.

Acid labile linkers include, but are not limited to, cis-aconitic acid, cis-carboxylic alkadienes, cis-carboxylic alkatrienes, and poly-maleic anhydrides. Other cleavable linkers are linkers capable of attaching to primary alcohol groups. Cleavable linkages may not only include linkages that are labile to reaction with a locally acting reactive species, such as singlet oxygen, but also include linkages that are labile to agents that operate throughout a reaction mixture, such as a base cleaving all base-labile linkages, general illumination by light of an appropriate wavelength cleaving all photocleavable linkages, and so on. Additional linkages cleavable by agents that act generally throughout a reaction mixture include linkages cleavable by reduction, linkages cleaved by oxidation, acid-labile linkages, peptide linkages cleavable by specific proteases, and the like. References describing many such linkages include Greene and Wuts, Protective Groups in Organic Synthesis, Second Edition (John Wiley & Sons, New York, 1991); Hermanson, Bioconjugate Techniques (Academic Press, New York, 1996); and Still et al, U.S. Pat. No. 5,565,324. Further examples of cleavable linkages are given in “Tagged microparticle compositions and methods,” U.S. 2003/0134333.

Poly-cis carboxylic acids are further described in U.S. Pat. No. 7,097,835. That patent discusses the conjugation of a toxin to an antibody, where the toxin is released from the antibody at the cellular target (in that case a vesicular transporter protein on the surface of a neuronal cell). However, given the present teachings, one may adapt cleavable linkages used in certain immunoconjugates for use in the present invention. The chemical linker is further defined as having a chemical linkage that is cleaved in an intracellular environment. It may include photocleavable linkers, redox sensitive linkers, and linkers that can be cleaved by application of electrical potential or current to a cell.

An example of a photocleavable linker is a group that contains an electron-rich carbon-carbon double bond. This bond is cleaved by singlet oxygen, which is generated by radiation. Construction of such a linker is further described in Ruebner et al., “A cyclodextrin dimer with a photocleavable linker as a possible carrier for the photosensitizer in photodynamic tumor therapy,” Proceedings of the National Academy of Sciences of the United States of America, Vol. 96, No. 26 (Dec. 21, 1999), pp. 14692-14693, disclosing a linker comprising a linker having the formula —S—CH═CH—S—.

An example of a redox linker besides —S—S— is —C—S. Suitable redox linkers may be adapted from those used in solid phase synthesis. See, e.g., Zheng, A.; Shan, D.; Wang, B. “A Redox-Sensitive Resin Linker for the Solid Phase Synthesis of C-Terminal Modified Peptides” J. Org. Chem. 1999, 64, 156-161. British Patent No. 2100458 discloses the use of sulphonamidophenol and sulphonamidonaphthol dye-releasing redox compounds, which release a diffusible dye on, heat development. British Patent No. 2100016 discloses the use of dye-releasing couplers, which, in combination with a reducing agent, release a diffusible dye on heat development. Various other dye-releasing systems have been disclosed, e.g., U.S. Pat. Nos. 4,060,420, 4,088,469, 4,499,180, 4,511,650, and 4,731,321, often involving thermal generation of a basic substance.

A cleavable linker may also be cleavable electrostatically, rather than chemically. That is, the payload may be attached to the nanostructure by applying a charge to the nanostructure, which is opposite to the charge that the payload possesses in solution. Charged molecules may be polynucleic acids, metal containing molecules, halogen containing molecules, poly amino acids containing charged residues, and so forth. Generally, the molecular payload will bear a negative charge, and the nanostructure will have a positive charge. For example, the resting potential for a ventricular myocyte is about −90 mV. Nerve cells have a resting potential of about −65 mV. A positive bias approximately 50 to 100 mV will be reduced inside the cell. A voltage source supplying a positive bias of about 5 volts, preferably 0.5 to 3 volts, can be used to attract negatively charged molecules. Further information about the negative charge of DNA may be found in Proc Natl Acad Sci U S A. 2002 Oct. 29; 99 (22): 14142-14146. The amount of the charge needed to attract the DNA in the solution to the nanostructure will depend on the ionic strength of the solution in which the tip is located.

### EXAMPLES

### Example 1

### Construction of A Nanomanipulation System

The nanomanipulation system (XYZ manipulator with imaging) was built based on a commercially available AFM (MFP-3D-BIO™, Asylum Research, Santa Barbara, Calif.) that integrates an inverted fluorescence microscope (Nikon TE2000). Recent biological applications of atomic force microscopy (14) have provided information on the integrity and local mechanical properties of cell membranes (4, 15-17). The spatial control mechanism of AFM piezo devices ensures the control of nanoneedle displacement at nanometer scale resolution. The sensitivity and ability to apply and monitor forces on a cell makes the AFM perfect nanomanipulator for penetration and withdraw of nanoneedles in this cell nanoinjection system (FIGS. 1B and C). The fabrication of CNT-AFM tips was carried out in an FEI Sirion XL 30 SEM, equipped with a homemade manipulator. The procedure was described in detail in a previous publication [Nanotechnology 16, 2493 (2005)].

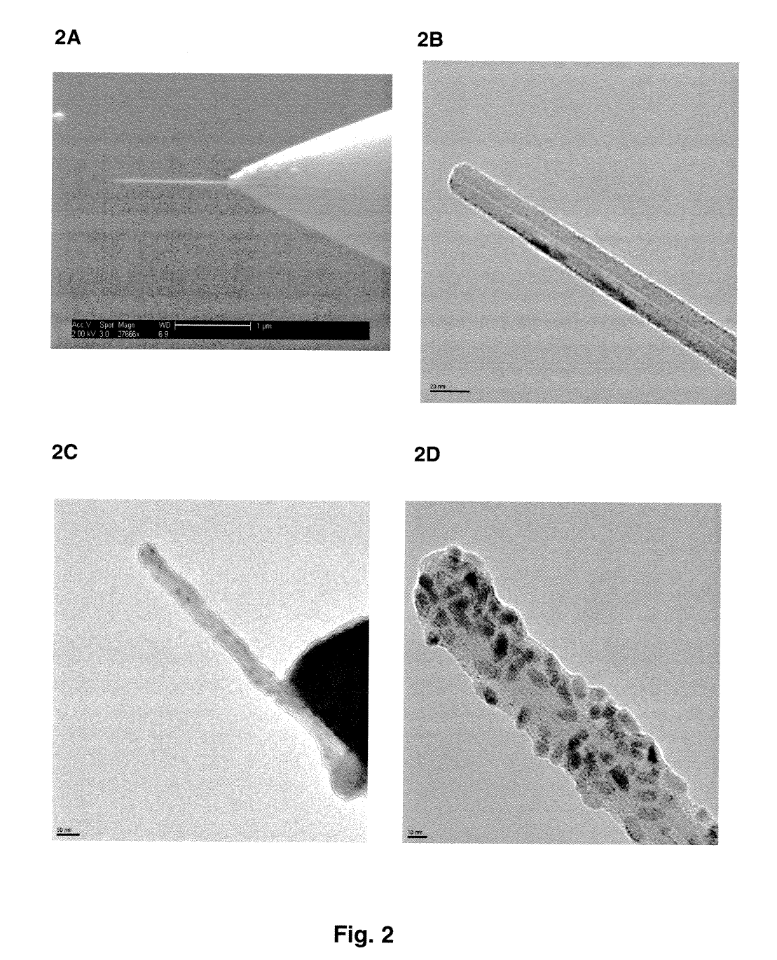

In brief, an individual multi-walled CNT of 10-20 nm in diameter was retrieved from a metal foil by the AFM tip using a nanomanipulator inside a scanning electron microscope (SEM). The CNT was then cut to the desired length (0.5-1.5 μm) using an electron beam or electrical current. SEM and transmission electron microscopy (TEM) images of one representative CNT-AFM tip are shown in FIGS. 2A and B, respectively.

### Example 2

### Attaching Nanoinjector To Microscopic Tip

To manipulate CNT nanoneedles, we first attached them at one end to AFM tips. Several methods have been reported for attaching CNTs to AFM tips (18-20). The CNT-AFM tips used in this work were fabricated as described previously (20). In brief, an individual MWCNT was retrieved from a metal foil by the AFM tip using a nanomanipulator inside a scanning electron microscope (SEM). As described in Ref. 20, multiwalled CNTs were prepared by the standard arc-discharge technique. After purification, a 10 μl ethanol suspension of CNTs was deposited between two metallic electrodes placed on a clean glass microscope slide, separated by a 400 μm gap. CNTs were aligned on the edge of the electrodes when a 70 V-1 kHz AC signal was applied across the gap. The metallic electrode with the CNTs was then mounted inside the SEM on the nanomanipulator with the CNTs positioned perpendicular to the plane of the AFM cantilever. SEM imaging allows us to control the motion of the AFM tip to bring it in contact with one CNT protruding from the edge of the electrode.

Cutting the nanotube by electron beam or electrical current gave the desired length, which in this case is between 0.5 and 1.5 μm. SEM and transmission electron microscope (TEM) images of one representative CNT-AFM tip are shown in FIG. 2A and 2B, respectively. The CNT-AFM tips typically have CNT nanoneedles of 10-20 nm in diameter and 0.5-1.5 μm in length.

SEM images of CNT-AFM tips were obtained on an FEI Sirion XL 30 SEM operated at 5 keV. TEM images of unfunctionalized and functionalized CNT-AFM tips were obtained on a JEOL 2011 microscope operating at an electron energy of 100 keV. A homemade holder was used for loading CNT-AFM tips.

### Example 3

### Synthesis of Linker And Attachment of A Protein

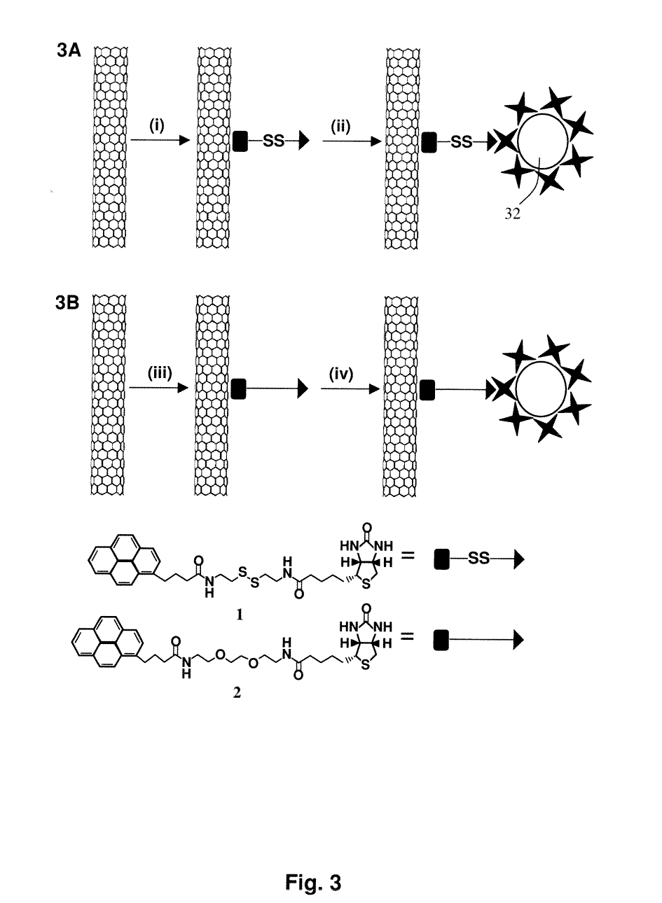

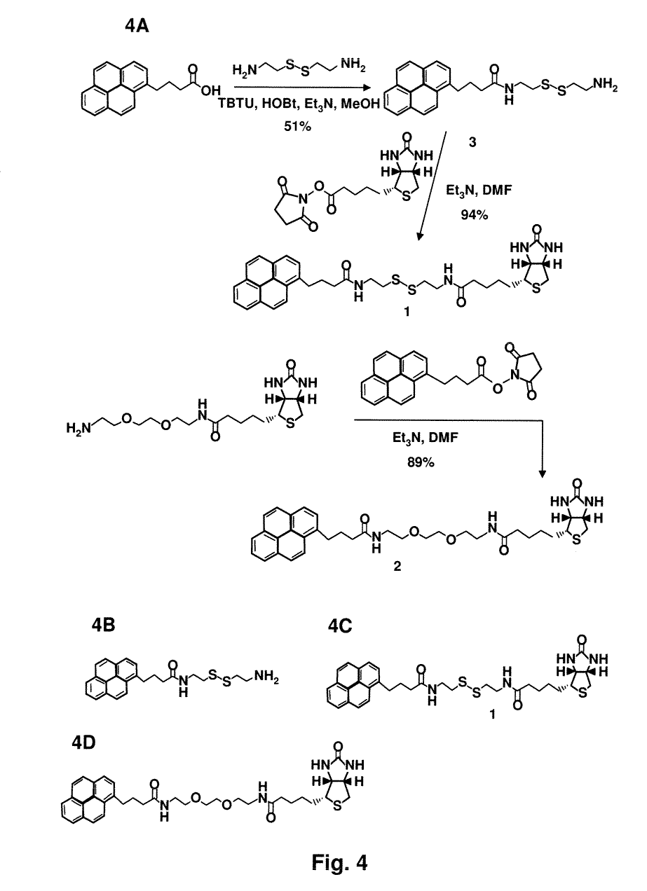

For the controlled loading and release of cargo, we aimed to design a system that would obviate the need for a carrier solvent and, accordingly, the addition of excess volume to the cell's cytosol during the injection process. Toward this end, we exploited established chemical methods for CNT surface modification and the intrinsic difference in redox potential between the intracellular and extracellular environments. Compound 1 (FIG. 3) fulfilled the functions of cargo loading and release as follows. Its pyrene moiety binds strongly to CNT surfaces via π-π stacking (12). Compound 1 is also endowed with a biotin moiety, separated from the pyrene group via a disulfide bond. In the relatively oxidizing environment of the cell's exterior, the disulfide is stable. However, once exposed to the reducing environment of the cytosol, the disulfide is cleaved, liberating attached cargo. The kinetics of disulfide bond cleavage within mammalian cells has been extensively studied, allowing prediction of release rates during the nanoinjection process.

We then explored the power of the nanoinjector by nanoinjecting proteins into cells. Streptavidin, a protein with clinical applications in anticancer therapies, was chosen to be nanoinjected into HeLa cells. See, Schultz et al., “A Tetravalent Single-chain Antibody-Streptavidin Fusion Protein for Pretargeted Lymphoma Therapy,” Cancer Research 60, 6663-6669, Dec. 1, 2000.

In order to visualize streptavidins in the cells after nanoinjection, QDot® semi-conductor nanocrystal -Streptavidin conjugates (Quantum Dot Corp., Hayward, Calif.) were used. A tri-functional linker (Compound 1, FIG. 3), which contains a pyrene moiety at one end, a biotin moiety at the other end, and a disulfide bond in between, was designed and synthesized to conjugate semi-conductor nanocrystal streptavidin conjugates onto CNT surface.

As described in Austin et al., “Oxidizing potential of endosomes and lysosomes limits intracellular cleavage of disulfide-based antibody-drug conjugates,” Proc. Natl. Acad. Sci. 102 (50) 17987-17992 (Dec. 13, 2005), current antibody-drug conjugate designs incorporate a disulfide linker between the antibody and cytotoxic drug are inspired by indirect evidence suggesting that the redox potential within the endosomal system is reducing. Such linkers include hydrazone linkers, designed to hydrolyze upon internalization into acidic endosomes and lysosomes; peptide linkers optimized for cleavage by certain lysosomal proteases; and disulfide linkers, thought to be cleaved by the reducing environment within the endocytic pathway. Examples of antibody-toxin linkers are a 4-succinimdyloxycarbonyl-methyl-{alpha}[2-pyridyldithio]-toluene (SMPT) linker and an N-succinimidyl 4-(2-pyridyldithio) pentanoate (SPP) linker. The authors further report that proteolysis is likely the predominant mechanism of dequenching (cleavage of SPP). They observed that that endosomes and lysosomes were oxidizing.

Pyrenes have strong interactions with CNT surface via π-π stacking (21) and biotins bind to steptavidins with dissociation constant on the order of 10-15 M. A disulfide bond is a covalent linkage, which arises as a result of the oxidation of two sulfhydryl groups, and can reversibly be cleaved in the presence of reducing agents. The presence of a high redox potential difference between the oxidizing extracelluar space and the reducing intracellular space (cytosol) makes the disulfide bond intriguing for a controlled cleavage and release of cargoes upon cell entry (13). Thus, semi-conductor nanocrystal streptavidin conjugates are loaded onto CNT surface via linker 1 and released in the reducing cytosolic space.

### Materials And Methods

QDot® 655 streptavidin conjugates (1 μM solution, purchased from Invitrogen) were centrifuged at 5,000×g, reserving the supernatant, prior to use. The CNT-AFM tips were incubated with linker 1 or 2 (1 μM, MeOH) at rt for 1 h, followed by washing 3 times with methanol and borate buffer (50 mM, pH=8.3), respectively. The CNT -AFM tips functionalized with Compounds 1 or 2 were then incubated with blocking buffer (borate buffer containing 1% BSA) for 30 min. The blocked CNT -AFM tips were then transferred (see following example) to a solution of semi-conductor nanocrystal streptavidin conjugates (1:25 dilution) in borate buffer and incubated at rt for 30 min, followed by washing with borate buffer for 3 times. The functionalized CNT-AFM tips were then used directly for nanoinjection experiments or dried under N2 for TEM characterization, and as described in Example 5.

All chemical reagents were of analytical grade, obtained from commercial suppliers and used without further purification. Flash chromatography was performed using Merck 60 Å 230-400 mesh silica gel. Analytical TLC was performed on Analtech Uniplate silica gel plates and visualized by staining with ceric ammonium molybdate or by absorbance of UV light at 254 nm. 1HNMR, and 13CNMR syectra were obtained with Bruker AMX-400 or Bruker DRX [symbol] 500 MHz spectrometers. IH and 3C chemical shifts (δ) are reported in parts per million (ppm) referenced to TMS (0 ppm) and were measured relative to the residual solvent peak. Coupling constants (J) are reported in hertz (Hz). High-resolution fast atom bombardment (F AB) mass spectra were obtained at the UC Berkeley Mass Spectrometry Laboratory.

N-((pyren-1-yl)butanoyl)cystamine (compound 3). To a solution of cystamine dihydrochloride (1.00 g, 4.40 mmol) and 1-pyrenebutyric acid (1.27 g, 4.40 mmol) in 40 mL of anhydrous MeOH were added TBTU (2.80 g, 8.80 mmol), HOBt (0.890 g, 6.60 mmol), and triethylamine (1.82 mL, 13.2 mmol). The reaction mixture was stirred at rt overnight. The solvent was evaporated, and 1 M NaH₂PO₄ was added (10 mL, pH=4.2). The aqueous was washed with ether. The aqueous solution was then basified to pH=9 by 10 M NaOH and extracted with EtOAc (5 mL×6). The combined organic phases were dried over MgSO₄, filtered, and concentrated. The resulting residue was purified by silica gel column chromatography eluted with MeOH:CH₂Cl₂ (1:4) to give the product (950 mg, 51%). Rf=0.40 (MeOH:CH₂Cl₂=1:4). ^{I}H NMR (CD₃0D, 400 MHz) (j8.13 (d, J=9.3 Hz, 1 H), 8.02 (d, J=7.5 Hz, 2H), 7.94 (d, J=8.4 Hz, 2 H), 7.84-7.76 (m, 3 H), 7.70 (d, J=7.8 Hz, 1 H), 3.43 (t, J=6.6 Hz, 2 H), 3.20 (t, J=7.5 Hz, 2 H), 2.8 (t, J=6.3 Hz, 2 H), 2.71 (t, J=6.6 Hz, 2 H), 2.65 (t, J=6.3 Hz, 2 H), 2.25 (t, J=7.2 Hz, 2 H), 2.10-1.98 (m, 2 H). ¹³C NMR (CD30D, 100 MHz) (j 176.0, 137.3, 132.8, 132.3, 131.3, 129.9, 128.6, 128.5, 128.4, 127.7, 127.0, 126.2, 126.1, 126.0, 125.8, 124.4,41.8,41.2, 39.6, 38.6, 36.8, 33.8, 29.0. FAB-HRMS calcd for CZ4Hz7NZOSZ [MH]+: m/z 423.1565; found 423.1562.

N-(pyren-1-yl)butanoyl-N′-(biotinyl)cystamine (compound 1). To a solution of biotin N-hydroxysuccinimide (290 mg, 0.850 mmol) in 4 mL of DMF were added N-((pyren-1-yl)butanoyl)cystamine (360 mg, 0.850 mmol), and triethylamine (0.350 mL, 2.55 mmol). The reaction mixture was stirred at rt for 7 h. DMF was then removed under high vacuum. The resulting residue was purified by silica gel column chromatography eluting with MeOH:CH₂Cl₂ (1:9) to give the product (520 mg, 94%). Rf=0.64 (MeOH:CH₂Cl₂=1:4). ^{I}H NMR (CDCl₃+CD₃OD₃, 500 MHz) δ 8.14 (d, J=9.0 Hz, 1 H), 8.04-7.98 (m, 2 H), 7.96 (d, J=8.0 Hz, 2 H), 7.89-7.81 (m, 3 H), 7.72 (d, J=7.5, 1 H), 4.22-4.17 (m, 1 H), 3.99-3.93 (m, 1 H), 3.37 (t, J=6.5 Hz, 2 H), 3.33 (t, J =6.6 Hz, 2 H), 3,27-3.23 (m, 2H), 2.82-2.77 (m, 1 H), 2,68-2.63 (m, 4 H), 2.61 (dd, J=12.5,4.5 Hz, 1 H), 2.50 (d, J=12.5 Hz, 1 H), 2.22 (t, J=7.5 Hz, 2 H), 2.11-1.98 (m, 4 H), 1.49-1.35 (m, 4 H), 1.21-1.09 (m, 2 H). 13C NMR (CDCb+CD30D, 125 MHz) (j 174.5, 174.2, 164.0, 135.9, 131.3, 130.8, 129.9, 128.6, 127.4, 127.3, 126.7, 125.9, 125.0, 124.8(9), 124.8(6), 124.8, 124.7, 123.3,62.0, 60.3, 55.4, 53.4, 40.1, 38.4, 38.3, 37.7, 37.5, 35.8, 35.5, 32.8, 28.2, 27.9, 27.5, 25.4. FAB-HRMS calcd for C₃₄H₄₁N₄O₃S₃ [MH]+: m/z 649.2341; found 649.2323.

N-(pyren-1-yl)butanoyl-N′-biotinyl-3,6-dioxaoctane-1,8-diamine (compound 2). To a solution of 1pyrenebutyric acid N-hydroxysuccinimide (55.0 mg, 0.140 mmol) in 2 mL of DMF were added EZ-Link® Biotin-PEG₂₋Amine (Pierce, Ill.) (50.0 mg, 0.130 mmol), and triethylamine (20.0 μL, 0.140 mmol). The reaction mixture was stirred at rt for 5 h. DMF was then removed under high vacuum. The resulting residue was purified by silica gel column chromatography eluting with MeOH:CH₂Cl₂ (15:85) to give the product (75.0 mg, 89%). Rf=0.62 (MeOH: CH₂Cl₂=1:4). ¹H NMR (CDCl₃, 400 MHz) δ 8.25 (d, J=9.2 Hz, 1 H), 8.11 (dd, J=7.6,2.4 Hz, 2 H), 8.05 (d, J=8.8 Hz, 2 H), 7.97-7.92 (m, 3 H), 7.81 (d, J=7.6, 1 H), 6.67 (br, 2 H, NH), 6.57 (br, 1 H, NH), 5.72 (br, 1 H, NH), 4.21-4.18 (m, 1 H), 3.96-3.93 (m, 1 H), 3.503.27 (m, 14 H), 2.84-2.78 (m, 1 H), 2.63 (dd, J=12.8,4.4 Hz, 1 H), 2.56 (d, J=11.6 Hz, 1 H), 2.28 (t, J=7.2, 2 H), 2.19-2.13 (m, 2 H), 2.08 (t, J=7.2, 2 H), 1.58-1.43 (m, 4 H), 1.28-1.17 (m, 2 H). 13C NMR (CDCb, 100 MHz) D 173.6, 173.3, 164.4, 136.2, 131.5, 131.0, 130.0, 128.9, 127.7, 127.6, 127.5, 126.8, 126.1, 125.2, 125.1, 125.0, 125.9, 123.6, 70.2, 70.1, 70.0, 69.9, 61.8, 60.3, 5.7, 50.8, 40.5, 39.4, 39.2, 36.0, 32.9, 28.3, 28.1, 27.6, 25.7. FAB-HRMS calcd for C₃₆H₄₅N₄O₅S [MH]+: m/z 645.3111; found 645.3092.

### Example 4

### Loading Protein Conjugates Onto An MWNT

To demonstrate the function of the nanoinjector, we sought to deliver quantum dots to the cell's cytosol without concomitant membrane and cell damage, effects that are hard to avoid with conventional delivery technologies (1). Quantum dots have emerged as powerful optical probes for single particle and single molecule studies in cellular systems (15). Their bright fluorescence and resistance to photobleaching have enabled single-particle tracking of membrane proteins on the cell surface (16) and vesicles within cells (17). Without a delivery vehicle, quantum dots cannot access the cell's cytosol and nuclei. Accordingly, processes therein have been refractory to study using quantum dot technology.

We coated the CNT-AFM tip with compound 1 by co-incubation in methanol. The tip was then loaded with streptavidin-coated quantum dots (QDot® Streptavidin, Invitrogen) via non-covalent complexation of streptavidin with biotin in borate buffer (FIG. 3A). The quantum dots are shown at 32 as small “X”s attached to a large sphere which represents a molecule of streptavidin. The loaded CNT-AFM tips were characterized by TEM. As shown in FIG. 2C, multiple quantum dot streptavidin conjugates were successfully loaded onto a single CNT functionalized with compound 1 (up to several hundred per 1-μM tip). FIG. 3A shows a schematic structure of a disulfide cleavable linker attached to the quantum dot/streptavidin conjugate, while FIG. 3B shows the steps involved in creating a similar, non-cleavable linker, which was used as a control.

The loaded CNT-AFM tips were characterized by TEM. As shown in FIGS. 2 C and D, semi-conductor nanocrystal -streptavidin conjugates were successfully attached onto CNT surface functionalized with linker 1.

### Example 5

### Nanoinjection of Conjugated Protein Into Cultured Human Cells

The nanoinjection experiments were then carried out in cultured Hela cells. HeLa cells were grown in DMEM supplemented with penicillin (100 unit/mL), streptomycin (0.1 mg/mL), and 10% FCS and maintained in a 5% C02, water-saturated atmosphere at 37° C.

A target cell within the field was identified microscopically, using combined bright-field and fluorescence imaging. The cantilever was then positioned on top of the target cell, and the scan size was set to 0 nm. The deflection of the cantilever was measured with a photodiode to monitor the displacement of nanoneedle when the CNT-AFM was approaching the cell surface. After the tip of CNT came into contact with the cell, the cantilever was further lowered so that the CNT nanoneedle penetrated the cell. The CNT nanoneedle was then kept inside the cell for a period of time to allow at least a portion of the disulfide bonds to be reduced and the quantum dot (semi-conductor nanocrystal) streptavidin conjugates to be released into cytosol. Immediately after injection, the cantilever was withdrawn and the cell was observed under fluorescence microscope. Several semi-conductor nanocrystal-streptavidin conjugates were observed in the targeted cell after nanoinjection. Release of semi-conductor nanocrystal -streptavidin conjugates was observed normally after keeping CNTs inside the cytosol for more than half an hour. This slow reaction rate might be due to the multivalency of steptavidins and more than one steptavidins per quantum dot. In the present example, the semi-conductor nanocrystal-streptavidin conjugates were immobilized in the cell. It's also typical to observe free moving semi-conductor nanocrystal-streptavidin conjugates confined inside the target cell after injection, which would finally immobilize probably by attaching to membranes.

Fluorescence intensity inside the target cell indicated the release of quantum dots. Quantum dot streptavidin conjugates were never observed in neighboring cells. We confirmed that the released quantum dots were within the cell's interior by video microscopy analysis. Their mobility was limited to the confines of the cell, where they exhibited slow diffusion and eventual immobilization, perhaps due to interactions with organelle membranes or cytoskeletal fibers. In addition, we demonstrated that a single functionalized nanoneedle can be used for successive nanoinjection of multiple cells.

In a typical experiment, a 15 to 30-minute incubation of the nanoneedle inside the cell was sufficient for release of a detectable number of quantum dots. This observation is consistent with published disulfide reduction rates. Each quantum dot possesses ˜15 streptavidin molecules and each streptavidin molecule can bind four biotin moieties. Therefore, the quantum dots are likely bound to CNT surfaces via multiple disulfide bonds. The complete reduction of four disulfides within a protein molecule requires 15 minutes to 1 hour, consistent with the release kinetics that we observe in situ. Based on fluorescence intensity calibration experiments using free quantum dots in solution, and the sensitivity of our fluorescence microscope, we estimate that the fluorescence intensity observed represents small clusters of quantum dots with a diameter of 50-100 nm (i.e., 5-50 quantum dots depending on their arrangement).

### Example 6

### Control Experiment With No Cleavable Linker

In a control experiment, CNT-AFM tips were incubated directly with QDot® Streptavidin without prior coating with linker compound 1. A TEM image of a CNT-AFM tip before incubation with semi-conductor nanocrystal-streptavidin conjugates showed a symmetrical tube with a smooth surface (data not shown). A TEM image of the CNT-AFM tip after incubation with semi-conductor nanocrystal- streptavidin conjugates showed approximately the same image, because in this case, no QDot® semi-conductor nanocrystal streptavidin conjugates were observed on the CNT surface.

To confirm the releasing mechanism of nanoinjection of semi-conductor nanocrystal-streptavidin conjugates, we synthesized another linker (2, FIG. 3B) in which the disulfide bond was replaced with a PEG group. We then functionalize CNT-AFM tip with 2 followed by incubation with QDot® semi-conductor nanocrystal streptavidin conjugates. The semi-conductor nanocrystal streptavidin conjugates were loaded on CNT-AFM tips in a similar manner confirmed by TEM.

The CNT-AFM tips were incubated with blocking buffer for 30 mm. The blocked CNT-AFM tips were then transferred to a solution of QDot® 655 semi-conductor nanocrystal streptavidin conjugates (1:25 dilution) in borate buffer and incubated at rt for 30 min, followed by washing 3 times with borate buffer. The CNT-AFM tips were then dried under N₂ for TEM characterization.

The same nanoinjection experiments were carried out in Hela cells and no streptavidin-QD conjugates were observed under fluorescence microscope after injection. These results showed that the semi-conductor nanocrystal streptavidin conjugates were released selectively by disulfide bond reduction in the reducing cytosolic space.

In the control experiments in which semi-conductor nanocrystal streptavidin conjugates were loaded onto the CNTs using linker 2 which possesses pyrene and biotin moieties but lacks a disulfide bond using HeLa cells, no semi-conductor nanocrystal streptavidin conjugates were released (with >5 different CNT-AFM tips in >10 injection experiments).

In combined bright-field and fluorescence image of another four examples of HeLa cells after nanoinjection of semi-conductor nanocrystal streptavidin, none of which fluorescent semi-conductor nanocrystal streptavidin conjugates were released. In all cases, fluorescence images were acquired with λex=415 nm and data collection was done with a 655 nm filter.

Functionalization of CNT-AFM tips with QDot® Streptavidin conjugates via linker compound 2 also was studied in a TEM image of the CNT-AFM tip after functionalization with 2 and then conjugated with semi-conductor nanocrystal streptavidin. The resulting CNT-AFM tip appeared similar to CNT-AFM tips bearing the disulfide-bound conjugates.

### Example 7

### Cell Viability Measurements

A limitation of many intracellular delivery technologies is the harmful effects they exert on membranes and cells. Therefore, we probed the effects of nanoinjection on membrane integrity and cell viability using three assays: (i) the typan blue exclusion assay, (ii) the Calcein AM assay, and (iii) the Annexin V-FITC/propidium iodide (PI) assay for apoptosis. In the typan blue assay, the dye was added immediately after cell nanoinjection and the cells were monitored for 10 hours thereafter. No typan blue inclusion or reduction in cell viability was observed during this time period (Table 1).

TABLE 1
Typan blue test of cells after nanoinjection

| | Typan blue test |
|---|---|
| Cells after nanoinjection of the QDot ® Streptavidin | Unstained (alive) |
| conjugates | |
| Cells penetrated with nanoneedle without loading of | Unstained (alive) |
| cargo | |
| Cells penetrated with nanoneedle loaded with | Unstained (alive) |
| QDot ® Streptavidin conjugates using linker 2 | |
| Positive control: cells penetrated with AFM tips with | Stained (dead) |
| micron scale tip | |

In the Calcein AM assay, the cells were loaded with the fluorescent dye immediately prior to nanoinjection. Similar to the previous results, we saw no evidence of compromised membrane integrity for up to 10 hours. Finally, nanoinjected cells showed no detectable staining with Annexin V-FITC or PI up to 10 hours after the event. Thus, nanoinjection does not appear to induce apoptotic pathways in the cells. In some experiments we held the nanoneedle inside the cell for >1 hour without any visible effect on membrane integrity and cell viability. By contrast, a microinjection needle must typically be retracted within seconds of injection in order to avoid cell damage (J. C. Lacal, R. Perona, J. Feramisco, Microinjection (Birkhauser Verlag, 1999). The biocompatibility of nanoinjection will enable for exploration of a broad range of release chemistries that occur over extended time periods.

### Example 8

### Monitoring Intracellular Diffusion

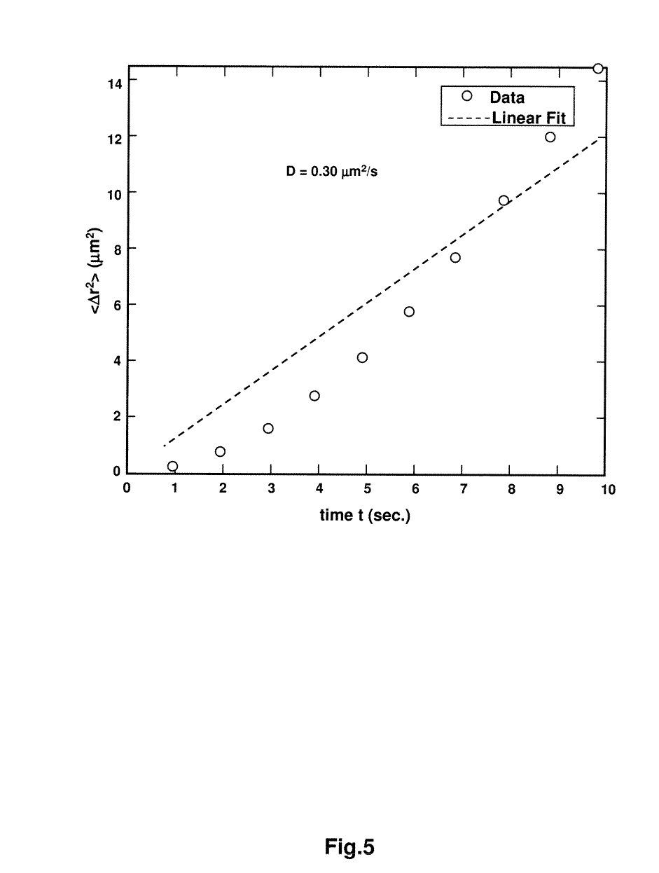

The ability to deliver quantum dots to the cell's cytoplasm provides a platform for numerous studies of intracellular processes. As an example, we used the single-particle tracking technique ((M. Dahan et al., Science 302, 442 (Oct. 17, 2003), X. L. Nan, P. A. Sims, P. Chen, X. S. Xie, J. Phys. Chem. B 109, 24220 (2005) to characterize the diffusion dynamics of injected quantum dots in the cytosol, which has been previously studied using methods that can harm cells. After nanoinjection, the diffusion dynamics of cytosolic quantum dots were characterized by analyzing the mean square distance (Δr²) and traveling time (Δt) for an injected quantum dot cluster (FIG. 5). The slope of the best-fit line afforded a diffusion coefficient of 0.3 μm²/sec. This value is approximately 10-fold lower than diffusion coefficients measured in pure water, which is consistent with previous measurements. A major advantage of the biocompatible nanoinjection technology is that the process can be performed repeatedly, or in tandem with other measurements, throughout the normal life cycle of the cell and monitor localization of injected materials.

### Example 9

### Electrostatic Linker

This embodiment of the present invention involves the attachment of a negatively charged payload to the nanostructure (e.g., MWNT), which is puncturing the cell membrane. HeLa cells were cultured in a Petri dish and a plasmid encoding green fluorescent protein (GFP) was added to the cell culture media. An MWNT nanoinjector was made as described above. A bias of 0.3 to 0.5 volts was applied to the MWNT so that the MWNT carried a positive charge, which attracted the DNA plasmids on to the nanostructure surface.

The MWNT was then manipulated to pierce the cell membrane. Once inside the cell membrane, the bias was reversed to a negative potential so that the DNA was repelled away from the nanotube surface, resulting in release of the plasmid DNA into the cell.

The cells were then cultured for three additional days and examined for expression of GFP, meaning that the cells had taken up the plasmid as a result of the insertion of the MWNT.

It is believed that the DNA, having a negative charge, was attracted to the positive charge on the MWNT, and thus was carried with the MWNT as it entered the cell. Once inside the cell, it is believed that the DNA is released by the dissipation of the electrostatic charge on the MWNT. This is brought about by the negative charge of the cell's cytoplasm.

When attracting a positively charged molecule to the MWNT, one would begin with a negative bias, then reverse to a positive bias inside the cell.

### CONCLUSION

Thus there is described a cell nanoinjector based on nanostructures such as CNTs. The results described here demonstrate that this nanoinjector system can be used to nanoinject biomolecules such as proteins into mammalian cells. Other molecules (such as plasmid DNA or small molecules) could be nanoinjected in similar manner. Another application of the present nanoinjector is to nanoinject cells with small size, for example, bacteria. Furthermore, the capability of AFM to image at nanoscale resolution may be utilized to target small organelles for injection. The injection may be carried out on a preselected, relatively exact number of molecules, even down to a single molecule and/or a single cell, by controlling the functionalization chemistry on the nanoinjector. The above specific description is meant to exemplify and illustrate the invention and should not be seen as limiting the scope of the invention, which is defined by the literal and equivalent scope of the appended claims. Any patents or publications mentioned in this specification are indicative of levels of those skilled in the art to which the patent pertains and are intended to convey details of the invention which may not be explicitly set out but which would be understood by workers in the field. Such patents or publications are hereby incorporated by reference to the same extent as if each was specifically and individually incorporated by reference, as needed for the purpose of describing and enabling the method or material referred to.

### REFERENCES

1. M. A. Barber, Journal of Infectious Diseases 8, 348 (April 1911).
2. J. C. Lacal, R. Perona, J. Feramisco, Microinjection (Birkhauser Verlag, 1999), pp.
3. G. M. Whitesides, Nature Biotechnology 21, 1161 (October 2003).
4. A. E. Pelling, S. Sehati, E. B. Gralla, J. S. Valentine, J. K. Gimzewski, Science 305, 1147 (Aug. 20, 2004).
5. N. a. Kouklin, W. E. Kim, A. D. Lazareck, J. M. Xu, Applied Physics Letters 87 (Oct. 24, 2005).
6. I. Obataya, C. Nakamura, S. Han, N. Nakamura, J. Miyake, Nano Letters 5, 27 (January 2005).
7. W. Z. Wu et al., J Am Chem Soc 127, 8914 (Jun. 29, 2005).
8. H. Andersson, A. van den Berg, Curr Opin Biotechnol 15, 44 (February 2004).
9. M. F. Yu, B. S. Files, S. Arepalli, R. S. Ruoff, Phys Rev Lett 84, 5552 (Jun. 12, 2000).
10. M. F. Yu et al., Science 287, 637 (Jan. 28, 2000).
11. Y. Lin et al., Journal of Materials Chemistry 14, 527 (Feb. 21, 2004).
12. M. C. Garnett, Adv Drug Deliv Rev 53, 171 (Dec.17, 2001).
13. G. Saito, J. A. Swanson, K. D. Lee, Adv Drug Deliv Rev 55, 199 (Feb. 10, 2003).
14. G. Binnig, C. F. Quate, C. Gerber, Physical Review Letters 56, 930 (Mar. 3, 1986).
15. C. Rotsch, M. Radmacher, Langmuir 13, 2825 (May 14, 1997).
16. E. A-Hassan et al., Biophysical Journal 74, 1564 (March 1998).
17. P. C. Zhang, A. M. Keleshian, F. Sachs, Nature 413, 428 (Sep. 27, 2001).
18. H. J. Dai, J. H. Hafner, A. G. Rinzler, D. T. Colbert, R. E. Smalley, Nature 384, 147 (Nov. 14, 1996).
19. J. H. Hafner, C. L. Cheung, A. T. Woolley, C. M. Lieber, Progress in Biophysics & Molecular Biology 77, 73 (2001).
20. J. Martinez et al., Nanotechnology 16, 2493 (November 2005).
21. R. J. Chen, Y. Zhang, D. Wang, H. Dai, J Am Chem Soc 123, 3838 (Apr. 25, 2001).

What is claimed is:

1. Apparatus for injecting a molecule into a selected target cell, comprising:
(a) a tip attached to a mechanical scanning device for positioning the tip relative to the target cell and for moving the tip into the target cell;
(b) a nanostructure fixed on an end of the tip; and
(c) a cleavable linker on the nanostructure for attaching the molecule to the nanostructure by the cleavable linker, said cleavable linker having a cleavable linkage which is cleaved in an intracellular environment to release the molecule.

2. The apparatus of claim 1 wherein the molecule is selected from the group consisting of: proteins, nucleic acids and small molecules.

3. The apparatus of claim 2 wherein said proteins and nucleic acids further comprise optical labels attached thereto.

4. The apparatus of claim 1 wherein said mechanical scanning device is a scanning probe microscope.

5. The apparatus of claim 4 wherein said scanning probe microscope is an atomic force microscope.

6. The apparatus of claim 1 wherein the nanostructure is selected from the group consisting of a nanowire, nanorod and a carbon nanotube.

7. The apparatus of claim 6 wherein the carbon nanotube is an MWNT.

8. The apparatus of claim 6 wherein the cleavable linker is of the formula Ar—R—X—Y where Ar is attached to the nanostructure and is an aryl compound, R is an alkyl linker, X is a cleavable functionality, and Y is an alkyl-linked binding group for binding to the molecule to be delivered.

9. The apparatus of claim 8 where Ar is a polycyclic aromatic hydrocarbon.

10. The apparatus of claim 8 where Ar is anthracene, naphthalene or pyrene.

11. The apparatus of claim 8 where R is lower alkyl.

12. The apparatus of claim 10 where X is —S—S—, poly-cis-carboxylic acids, S—CH═CH—S—, peptide and hydrazone.

13. The apparatus of claim 12 where X is —S—S—.

14. The apparatus of claim 8 where Y comprises biotin.

15. The apparatus of claim 7 further comprising a circuit for applying a charge to the nanostructure to apply a bias voltage opposite of a charge on a payload.

16. A method for injecting a molecule into a target cell at a selected location, comprising the steps of:
(a) attaching a molecule to a nanostructure by linking the molecule to the nanostructure, said having a linkage which is cleaved in an intracellular environment.
(b) identifying a target cell at a fixed location;
(c) contacting the target cell with a mechanical scanning device for positioning the nanostructure relative to the target cell; and
(d) inserting the nanostructure and the molecule into the target cell for a time sufficient to cleave the cleavable linker and release the molecule without using a carrier fluid to deliver the molecule.

17. The method of claim 16 further comprising the step of using an optical microscope to image the target cell for delivering the biomolecules and/or using an AFM microscope to obtain an AFM image of the cell.

18. The method of claim 16 wherein said target cell is selected from the group consisting of prokaryotic cells, animal cells, plant cells and viruses.

19. The method of claim 16 wherein said injecting is injecting into an organelle.

20. The method of claim 19 wherein the organelle is a cell nucleus.

21. The method of claim 16 wherein the injecting comprises injecting a molecule selected from the group consisting of proteins, nucleic acids, and small molecules.

22. The method of claim 21 wherein said proteins and nucleic acids further comprise optical labels attached thereto.

23. The method of claim 16 further comprising the step of injecting with movement controlled by a scanning probe microscope.

24. The method of claim 23 wherein said scanning probe microscope is an atomic force microscope.

25. The method of claim 16 wherein the nanostructure is selected from the group consisting of a nanowire, nanorod and a carbon nanotube.

26. The method of claim 25 wherein the carbon nanotube is an MWNT.

27. The method of claim 16 wherein the cleavable linker is of the formula Ar—R—X—Y where Ar is an aryl compound, R is an alkyl linker, X is a cleavable functionality, and Y is an alkyl-linked binding group for binding to a biological molecule.

28. The method of claim 27 where Ar is a polycyclic aromatic hydrocarbon.

29. The method of claim 27 where Ar is anthracene. naphthalene or pyrene.

30. The method of claim 29 where R is lower alkyl.

31. The method of claim 27 where X is —S—S—.

32. The method of claim 27 where Y comprises biotin.

33. The method of claim 16 further comprising the steps of applying a charge to the nanostructure opposite to a charge on the molecule to be delivered, so that the charge serves to link the molecule to the nanostructure by electrostatic force; and reversing the charge within the cell to deliver the molecule.

34. A method of making a nanostructure for injecting a payload molecule into a cell, comprising:
(a) providing a nanostructure consisting essentially of a hydrophobic material;
(b) preparing a cleavable linker of the formula Ar—R—X—Y where Ar is an aryl compound, R is an alkyl linker, X is a cleavable functionality, and Y is an alkyl-linked binding group for binding to a biological molecule; and
(c) contacting the nanostructure with the cleavable linker to allow the Ar to adsorb onto the nanostructure.

35. The method of claim 34 wherein the nanostructure is selected from the group consisting of a nanowire, nanorod and a carbon nanotube.

36. The method of claim 34 wherein the carbon nanotube is an MWNT.

37. The method of claim 34 wherein Ar is a polycyclic aromatic hydrocarbon.

38. The method of claim 34 wherein Ar is anthracene, naphthalene or pyrene.

39. The method of claim 34 wherein where R is lower alkyl.

40. The method of claim 34 wherein X is —S—S—, poly-cis-carboxylic acids. S—CH═CH—S—, peptide and hydrazone.

41. A nanostructure for injecting a payload molecule into a cell, comprising:
(a) a nanostructure consisting essentially of a hydrophobic material;
(b) a cleavable linker of the formula Ar—R—X—Y where Ar is an aryl compound. R is an alkyl linker, X is a cleavable functionality, and Y is an alkyl-linked binding group for binding to a biological molecule; and
(c) the nanostructure has adsorbed thereto the cleavable linker through the Ar.

42. The nanostructure of claim 41 wherein the nanostructure is selected from the group consisting of a nanowire, nanorod and a carbon nanotube.

43. The nanostructure of claim 41 wherein the carbon nanotube is an MWNT.

44. The nanostructure of claim 41 wherein Ar is a polycyclic aromatic hydrocarbon.

45. The nanostructure of claim 41 wherein Ar is anthracene, naphthalene or pyrene.

46. The nanostructure of claim 41 wherein R is lower alkyl.

47. The nanostructure of claim 41 wherein X is —S—S—, poly-cis-carboxylic acids, S—CH═CH—S—, peptide and hydrazone.
